(19) United States
(12) Patent Application Publication
(10) Pub. No.: US 2011/0207226 A1
(43) Pub. Date: Aug. 25, 2011

(54) NON-INTEGRATING LENTI/ADENO-ASSOCIATED VIRUS HYBRID VECTOR SYSTEM

(75) Inventors: Yajin NI, Germantown, MD (US); Jenice G. D' Costa, Gaithersburg, MD (US); Ramdall K. Merling, Rockvilley, MD (US)

(73) Assignee: Virxsys Corporation, Gaithersburg, MD (US)

(21) Appl. No.: 13/059,952

(22) PCT Filed: Aug. 20, 2009
(86) PCT No.: PCT/US09/54378

§ 371 Date: May 16, 2011

Related U.S. Application Data
(60) Provisional application No. 61/090,357, filed on Aug. 20, 2008.

(51) Int. Cl. C12N 15/85

(52) U.S. Cl. 435/462; 435/320.1

(57) ABSTRACT

The present invention provides for a hybrid vector system for the purpose of therapeutic gene delivery where the system is used for a targeted integration of a therapeutic gene into a genome. The hybrid vector system comprises a hybrid vector made up of a non-integrating lentiviral vector and an adeno-associated vector, and a therapeutic gene.

### CROSS REFERENCE TO RELATED APPLICATIONS

This application claims priority to U.S. Provisional Application No. 61/090,357, filed Aug. 20, 2008, the contents of which are incorporated herein by reference in their entirety.

### FIELD OF THE INVENTION

This application is related to new methods and materials for targeted integration of genes or nucleic acids of interest into host genomes, and more particularly to methods and materials for targeted integration of genes or nucleic acids of interest into host genomes mediated by non-integrating lenti/adeno-associated virus (“NILV/AAV”) hybrid vector system.

### BACKGROUND OF THE INVENTION

Human immunodeficiency virus 1 (HIV-1) based lentiviral vectors (LVs) have been identified as useful candidates for gene delivery applications. Their capacity to package a relatively large size of DNA (up to 10 kb), as well as their ability to transduce a range of dividing and non-dividing cell types, make them desirable gene delivery vehicles. The genome of LVs integrates into host genome, and this promotes efficient, stable transgene expression in cells. However, integration occurs in a non-specific manner which can be deleterious as it could cause perturbations of gene expression in host cells. As a result insertional mutagenesis could occur and lead to malignant transformation.

Although a better safety profile of LVs compared with MLV-based retroviral vectors has been reported, insertional mutagenesis due to non-specific integration still raises concerns. Self inactivating (“SIN”) vectors are often considered safer than conventional vectors in terms of insertional mutagenesis, but construction of SIN vector producer cell line using transduction-based methods is not feasible. Lately, non-integrating lentiviral vectors (“NILV”) that provide long-term expression in non-dividing cells and tissues have been developed to avoid the risk of insertional mutagenesis. However, NILV provides only short lived gene expression in dividing cells, with the vector genomes ultimately being lost during successive cell divisions.

Likewise, adeno-associated virus 2 (AAV-2) can infect a wide range of host cells, where they can integrate primarily in a site-specific manner. Site-specific integration is mediated by Rep proteins, along with cis integrating elements of AAV-2 and host factors, at the AAVS1 site on human chromosome 19p13.4q. Due to the limitation in packaging capacity of AAV-2, the recombinant AAV vectors based on AAV-2 virus, have been developed that are gutted of all viral genes including those responsible for site-specific integration in order to accommodate larger size of exogenous DNA. These AAV vectors support long-term gene expression in both nondividing and dividing cells and tissues as a persistent episomal form and through random integration.

Several hybrid vector systems (e.g., Ad/AAV and HSV/AAV) using Rep-mediated site-specific integration of AAV-2 virus have been developed. Rep68/78 proteins however have been reported to inhibit replication of several viral as well as cellular genomes, and cause reduced vector titers. The improved designs of HSV/AAV hybrid vectors have significantly increased the efficiency of site-specific integration, with some data showing 70% site-specific integration. However, transient expression of Rep proteins in target cells from the hybrid vector genome as a requirement for site-specific integration remains an unresolved safety concern. Accordingly, a continuing and unmet need for an improved hybrid vector system having eliminated Rep protein expression in target cells still remains.

### SUMMARY OF THE INVENTION

Provided herein is a new hybrid vector system that combines desirable features of both LV and AAV vectors, while simultaneously eliminating (or reducing) their undesirable characteristics.

In certain embodiments the present invention provides a NILV/AAV hybrid vector system for site-specific insertion of a nucleic acid sequence into a host genome. The system comprises a hybrid LV/AAV transfer vector, an integration defective LV packaging system, a Vpr-Rep fusion protein and an AAVS1 site.

In certain embodiments the hybrid LV/AAV transfer vector comprises HIV-1 cis elements required for production and transduction of hybrid vector viruses and AAV-2 cis elements required for site-specific integration of hybrid vector viruses.

In certain embodiments the hybrid transfer vector comprises an expression cassette wherein said expression cassette consists of a prokaryotic or eukaryotic promoter that can be constitutive, inducible or tissue specific, a therapeutic gene or a reporter gene and a polyadenylation signal.

The integration defective LV packaging system provides elements required for packaging the hybrid transfer vector into viral particles that are capable of transducing the host cell. In certain embodiments the integration defective packaging system provides a HIV-1 GagPol containing any or a combination of class I point mutations such as D64V, D116N and E152A in Pol gene, that will encode an integration defective integrase and prevent non-specific integration of the hybrid transfer vector genome.

In certain embodiments, Rep68/78 of AAV-2 could be expressed as a fusion protein with HIV-1 Vpr protein and incorporated into the NILV/AAV hybrid vector virus via Vpr protein, followed by directing site-specific integration of gene expression cassette flanked by ITRs of AAV-2. Vpr protein incorporates the Rep68/78 protein into the NILV/AAV hybrid vector virus. In certain embodiments the HIV-1 Vpr protein could be mutated to eliminate functions such as G2 arrest and cell killing.

The NILV/AAV hybrid vector system combines certain advantages of both LV and AAV vectors. It is an advantage of the system whereby: the system can avoid non-specific insertion of undesirable LV vector cis-elements derived from HIV-1 virus; the system can direct site-specific integration of ITR-flanked gene expression cassette at the AAVS1 site which is known to not be associated with any malignant transformation events; the system can utilize LV vector production system to prepare helper virus free vector viruses; the system can retain the larger packaging capacity (10 kb) and highly efficient transduction potential of LV vectors especially in non-dividing cells; and the system can insert multiple copies of the gene expression cassette and maintain stable gene expression at the AAVS1 site.

In certain embodiments the present system provides a method for producing NILV/AAV hybrid vector virus for site-specific integration of a nucleic acid sequence in host genome.

In one embodiment, a hybrid vector system is provided for targeted integration of a nucleic acid of interest into a host cell comprising of a hybrid transfer vector, an integration defective packaging system, a Vpr-Rep fusion protein and an AAVS1 site.

In another embodiment, a method is provided for targeted integration of a nucleic acid of interest in a host cell comprising: transducing said host cell with a NILV/AAV hybrid vector system comprising of a hybrid transfer vector, an integration defective packaging system, a Vpr-Rep fusion protein and an AAVS1 site.

These and other aspects of some exemplary embodiments will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments without departing from the spirit thereof. Additional features may be understood by referring to the accompanying drawings, which should be read in conjunction with the following detailed description and examples.

### BRIEF DESCRIPTION OF THE FIGURES

The maps illustrated in the drawings are not drawn to scale, and the relative sizes of particular segments or functional elements are not necessarily proportional to the lengths (e.g., number of base pairs) of the corresponding genetic sequences.

FIG. 1 schematically illustrates the structure of LV/AAV hybrid transfer vector in accordance with an example embodiment hereof. “LTR” is a long terminal repeat. “Ψ” is a packaging signal. “PPT” refers to a polypurine tract, and “cPPT” refers to a central polypurine tract. “RRE” is a Rev response element. “IEE” is an integration efficiency element. “ITR” refers to an inverted terminal repeat. “pA” is a poly(A) signal. “TsPro” refers to tissue specific promoter.

FIG. 2 illustrates the cloning strategy and the plasmids used for construction of the hybrid transfer vector of the present invention. In the first step, synthesized 5′ITR of AAV-2 is ligated into HIV transfer vector VRX430 at the BamHI and AgeI sites to create intermediate construct VRX430.1. The synthesized p5IEE element of AAV-2 is ligated into the NcoI and AgeI site of VRX430.1 to create intermediate construct VRX430.2. In an embodiment, EF1 alpha or a tissue specific promoter is ligated into the NotI and NcoI site of VRX430.2 in the reverse orientation, while simultaneously removing GFP reporter gene, to create intermediate construct VRX430.3. The therapeutic gene or a reporter gene is ligated into the NheI and ClaI site upstream of the promoter in the reverse orientation, to create intermediate construct VRX430.4. The SV40 polyA signal is ligated upstream of the therapeutic or reporter gene in the ClaI site, to create intermediate construct VRX430.5. The synthesized 3′ITR of AAV-2 is ligated in the XhoI site, to create the final hybrid LV/AAV transfer vector construct.

FIG. 3A-3D is a schematic drawing of the structures of the four packaging plasmids necessary for the construction of a packaging cell line to produce the hybrid vector of the present invention and represents examples of packaging plasmids in accordance with example embodiments hereof “TRE” refers to tet responsive element, “U3 TAR” refers to truncated hybrid LTR, “SCMV” refers to simian cytomegalovirus, “SV40” refers to simian virus 40 and “TK” refers to thymidine kinase, are promoter elements. “mVpr-Rep68/78,” “tTA,” “Syn-Rev/Tat,” “Syn-GagPolD64V,” and “TPLVSV-G” are vector production genes. “Puro,” “Neo,” and “Hyg” are selection marker genes. Poly(A) signals are not depicted. FIG. 3E is a schematic drawing of the structures of one additional packaging plasmid containing all viral genes such as Rev/Tat, GagPolD64V, and VSV-G necessary for production and packaging of the hybrid vector of the present invention, which is alternatively used to produce the hybrid vector by transient transfection method. “CMV” refers to human cytomegalovirus, “EH” refers to EF1α and HTLV hybrid, are promoter elements. “GagPolD64V”, “RevIRESTat” and VSV-G, are vector production genes.

FIG. 4 illustrates the cloning strategy and plasmids used for the construction of pU3TARsynGagPolD64V packaging plasmid. The D64V mutation is introduced into the Pol gene of VRX581 (pPCR-synGagPol) by site directed mutagenesis. The region of the Pol gene containing the D64V mutation is cut out using AscI-NsiI and ligated in the AscI-NsiI site of VRX810 (pU3TAR2synGagPolSCMVNeo) to replace the wt region of Pol to create integration defective packaging construct pU3TAR2synGagPolD64V.

FIG. 5 illustrates the cloning strategy and plasmids used for construction of tetracycline regulatable pTRE-mVpr-Rep packaging plasmid. In the first step, mutations that remove the G2 arrest and cell killing functions of HIV-1 Vpr are created in the construct pCI-Vpr (VRX805) by site directed mutagenesis to create intermediate constructs VRX805.1 which will harbor different combinations of G2 arrest and cell killing mutations. mVpr gene is PCR amplified from VRX805.1 using primers that add AgeI-NotI sites at the 5′ and 3′ ends of the mVpr gene and delete the stop codon. In the second step, the pTRE-GFP construct is cut with AgeI and NotI to remove GFP and the mVpr PCR amplified fragment is ligated into the AgeI-NotI site to create intermediate construct pTRE-mVpr. In the third step, synthesized Rep68/78 gene of AAV2 is ligated in frame into the NotI-SphI site of pTRE-mVpr to create pTRE-mVpr-Rep68/78 fusion protein construct. In the final step, a 15 amino acid linker sequence if ligated into the NotI site between mVpr and Rep68/78 to create the final pTRE-mVpr-Rep68/78 fusion protein construct.

FIG. 6 illustrates the maps of the three existing packaging plasmids described in FIG. 3, pTREtTASVpuroTREsynRevTat (VRX845), pSCMVTPLVSVGTKHyg (VRX829), and pCMVGagPolD64VRRERevTatRzEHVSV-G(VRX1188). pCMVGagPolRRERevTatRzEHVSV-G (VRX577) which is the parent plasmid of VRX1188 and encodes a wild type integrase, is used to produce the LV/AAV hybrid vector viruses for introducing the hybrid vector genome into packaging cell line by transduction described below in FIG. 7.

FIG. 7 depicts the steps involved in creating a stable packaging and producer cell line for the production of NILV/AAV hybrid vectors. Viral genes required to package viral genomes (transfer vector), rev/tat (synRevTat plasmid), gag/polD64V (SynGagPolD64V plasmid), envelope protein VSVG (VSVG plasmid) and mVpr-Rep68/78 (mVpr-Rep plasmid) are co-transfected into 293 cells using calcium phosphate or cationic lipids or electroporation. Stable packaging cell line clones containing all 4 plasmids are screened and selected for resistance to the related selection makers. Integrating LV/AAV hybrid vector virus is made by cotransfecting hybrid transfer vector plasmid (VRX430.6) and wt integrase packaging plasmid (VRX577) containing all viral genes (required to make virus particles) into 293F cells. Integrating LV/AAV hybrid vector virus with wt integrase protein incorporated inside the viral particles are collected in the supernatant. The integrating LV/AAV hybrid vector virus is used to transduce the packaging cell line to generate a producer cell line that will contain the LV/AAV hybrid vector integrated into the cellular genome. The producer cell line will produce NILV/AAV hybrid vector virus with defective integrase and mVpr-Rep68/78 proteins incorporated into the viral particles.

FIG. 8 is a schematic depicting the mechanism of site-specific integration of a nucleic acid sequence into the AAVS1 site by NILV/AAV vector. Upon transduction of a host cell by the NILV/AAV vector, the Vpr-Rep68/78 fusion protein mediates site-specific integration of the gene expression cassette flanked by 5′ITR and 3′ITR into AAVS1 site on chromosome 19, by recognizing 5′ITR, 3′ITR and p5IEE of AAV-2. In contrast, non-specific integration of the gene expression cassette flanked by 5′LTR and 3′LTR of HIV-1 does not occur due to defective HIV-1 integrase.

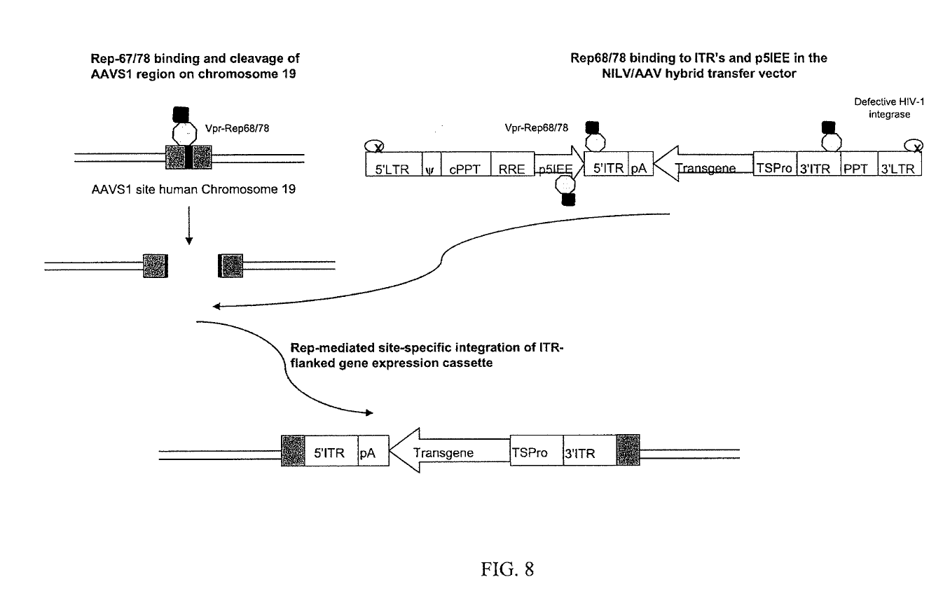

FIG. 9 illustrates the schematic diagram of the related plasmids used to examine Rep-mediated site-specific integration of hybrid LV-AAV vector plasmid by transient transfection experiment performed in Hela-tat cells.

FIG. 10 shows GFP expression from the hybrid LV-AAV vector plasmid and the related vector plasmids determined by flow cytometry on day 1 and day 13 after transfection. Hela-tat cells were doubly cotransfected using lipofectamine 2000 with VRX1090 control, LV-AAV2 GFP, ITR-GFP or ITR-p5IEE-GFP together with the second plasmid, control mock, Rep or Vpr-Rep.

FIG. 11 shows GFP expression of day 13 after transfection from the same experiment described in FIG. 10 by dot plot analysis.

### DESCRIPTION OF THE SEQUENCE LISTING

SEQ ID NO: 1 is AAV2 5′ITR: ACCESSION number:NC_{—}001401 REGION:1 . . . 145.

SEQ ID NO: 2 is AAV2 3′ITR: ACCESSION number:NC_{—}001401 REGION:4535 . . . 4679.

SEQ ID NO: 3 is AAV2 Rep68 protein: ACCESSION number:NC_{—}001401 REGION: join(321 . . . 1906,2228 . . . 2252).

SEQ ID NO: 4 is AAV2 Rep78 protein: ACCESSION number:NC_{—}001401 REGION:321 . . . 2186.

SEQ ID NO: 5 is p5IEE: ACCESSION number:NC_{—}001401 REGION:151-289

SEQ ID NO: 6 is HIV1 NL4-3 wt vpr: ACCESSION number:U26942 REGION: 4937 . . . 5227.

The following mutations singly or in combination can be used to create a mutant vpr (mVpr) protein that lacks the G2 arrest and cell killing ability: G2 arrest point mutations H78R, R80A and R88K; Cell killing point mutations: E24G, W54R. The sequences of the primers used to introduce the specific mutation into Vpr are as follows:

E24G:
(SEQ ID NO: 7)
5′ ggacactagagcttttaggggaacttaagagtgaagc 3′;
W54R:
(SEQ ID NO: 8)
5′ tctatgaaacttacggggatactagggcaggagtgg 3′;
H78R:
(SEQ ID NO: 9)
5′ tccatttcagaattgggtgtgcaagaagcagaataggcgttactcg
3″;
R80A:
(SEQ ID NO: 10)
5′ aattgggtgtcgacatagcgcaataggcgttactcgacag 3′;
and
R88K:
(SEQ ID NO: 11)
5′ ggcgttactcgacagaggaaagcaagaaatggagc 3′.

The sequence of the primers used to introduce the D64V mutation into the Pol gene to disrupt the integration function of integrase is as follows:

| (SEQ ID NO: 12) | |
|---|---|
| D64V | 5'gcatctggcaactggtctgcacacatctgga 3'. |

### DETAILED DESCRIPTION OF THE INVENTION

Provided herein is a new hybrid vector system that combines desirable features of both LV and AAV vectors, while simultaneously eliminating (or reducing) their undesirable characteristics.

The practice of the techniques described herein will employ, unless otherwise indicated, conventional techniques of cell biology, cell culture, molecular biology, genetics, microbiology, recombinant DNA, and immunology, which are within the skill of the art. The following illustrative explanations are provided to facilitate understanding of certain terms used frequently herein, particularly in the examples. The explanations are provided as a convenience and are not intended to be limiting.

As used in this specification and the appended claims, the singular forms “a,” “an,” and “the” include plural referents unless the content clearly dictates otherwise. Thus, for example, reference to “a vector” may include a combination of two or more vectors, reference to “DNA” may include mixtures of DNA, and the like.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are defined below.

A “virus” is an infectious agent that consists of protein and nucleic acid, and that uses a host cell's genetic machinery to produce viral products specified by the viral nucleic acid. A preferred RNA virus is a virus of the family Retroviridae (i.e., a retrovirus), particularly a virus of the genus or subfamily Lentivirus. A RNA virus of the subfamily Lentivirus is desirably a human immunodeficiency virus type 1 or 2 (i.e., HIV-1 or HIV-2, wherein HIV-1 was formerly called lymphadenopathy associated virus 3 (HTLV-III) and acquired immune deficiency syndrome (AIDS)-related virus (ARV)), or another virus related to HIV-1 or HIV-2 that has been identified and associated with AIDS or AIDS-like disease. The acronym “HIV” or terms “AIDS virus” or “human immunodeficiency virus” are used herein to refer to these HIV viruses, and HIV-related and -associated viruses, generically. Moreover, a RNA virus of the subfamily Lentivirus can be a Visna/maedi virus (e.g., such as infect sheep), a feline immunodeficiency virus (FIV), bovine lentivirus, simian immunodeficiency virus (SIV), an equine infectious anemia virus (EIAV), and a caprine arthritis-encephalitis virus (CAEV).

Another virus includes adeno-associated virus (AAV) which is a small non-pathogenic defective parvovirus which infects humans and other primates. The AAV genome of either plus or minus polarity is encapsidated as a single-stranded DNA with 4680 nucleotides. Replication of AAV requires coinfection with a helper virus of the herpesvirus or adenovirus family. Other parvoviruses replicate autonomously, but AAV requires co-infection with a helper virus such as adenovirus or herpes virus for lytic phase productive replication. In the absence of a helper virus, AAV establishes a latent, non-productive infection. AAV is not currently known to cause disease and consequently the virus causes a very mild immune response. AAV can infect both dividing and non-dividing cells and can incorporate its genome into that of the host cell at the AAVS1 site. AAV2 is one serotype for the invention, however other serotypes can be used as well, including, for example, and not by way of limitation, AAV1, AAV3, AAV4, AAV5, AAV6, AAV7, AAV8, AAV9, AAV10, and AAV11.

A “wild-type strain of a virus” is a strain that does not comprise any of the human-made mutations as described herein, i.e., any virus that can be isolated from nature. Alternatively, a wild-type strain is any virus that has been cultured in a laboratory, but still, in the absence of any other virus, is capable of producing progeny genomes or virions like those isolated from nature.

The term “host cell” or “target cell” refers to a cell transduced with a specified vector. The cell is optionally selected from in vitro cells such as those derived from cell culture, ex vivo cells, such as those derived from an organism, and in vivo cells, such as those in an organism.

A “vector” is a nucleic acid molecule (typically DNA or RNA) that serves to transfer a passenger nucleic acid sequence (i.e., DNA or RNA) into a host cell. Three common types of vectors include plasmids, phages and viruses. Preferably, the vector is a virus. The vector is not a wild-type strain of a virus, inasmuch as it comprises human-made mutations.

A vector also, preferably, is a “chimeric vector” or “hybrid vector”. Such vectors comprise a combination of sequences from a lentivirus with other sequences from AAV. Thus, the vector typically is derived from a wild-type strain of two different viruses (HIV-1 and AAV-2) by genetic manipulation (i.e., by deletion) to comprise cis and trans elements of LV and AAV vector to provide for site-specific integration of a transgene flanked by AAV cis elements without integration of LV cis elements.

The vector can also comprise some means by which the vector or its contained subcloned sequence is identified and selected. Vector identification and/or selection is accomplished using a variety of approaches known to those skilled in the art. For instance, vectors containing particular genes or coding sequences preferably are identified by hybridization, the presence or absence of so-called “marker” gene functions encoded by marker genes present on the vectors, and/or the expression of particular sequences. In the first approach, the presence of a particular sequence in a vector is detected by hybridization (e.g., by DNA-DNA hybridization) using probes comprising sequences that are homologous to the relevant sequence. In the second approach, the recombinant vector/host system is identified and selected based upon the presence or absence of certain marker gene functions such as resistance to antibiotics, thymidine kinase activity, and the like, caused by particular genes encoding these functions present on the vector. In the third approach, vectors are identified by assaying for a particular gene product encoded by the vector. Such assays are based on the physical, immunological, or functional properties of the gene product.

A “non-integrating lentiviral vector” is a vector with nucleic acid sequences derived from LV, which contains an integrase protein lacking integration activity, and hence cannot insert those nucleic acid sequences into the genome of a host cell. The non-functional integrase protein is expressed by a HIV-1 Pol gene that has been mutated specifically to destroy its integration ability.

The term “encapsidation” generically refers to the process of incorporating a nucleic acid sequence (e.g., a vector, or a viral genome) into a viral particle. No distinction is made between the type of nucleic acid and the type of viral particle. Thus, encapsidation refers to the process of placing a nucleic acid sequence (e.g., single-stranded RNA, double-stranded RNA, single stranded DNA and double-stranded DNA) into any type of viral particle, i.e., “capsid” is a generic term used to indicate any type of viral shell, particle or coat, including a protein capsid, a lipid enveloped structure, a protein-nucleic acid capsid, or a combination thereof (e.g., a lipid-protein envelope surrounding a protein-nucleic acid capsid).

The term “nucleic acid” refers to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides. Unless otherwise indicated, a particular nucleic acid sequence includes the complementary sequence thereof. A “nucleic acid” refers to a polymer of DNA or RNA that is single or double-stranded, linear or circular, and, optionally, contains synthetic, normatural, or modified nucleotides, which are capable of being incorporated into DNA or RNA polymers.

A “promoter” is a sequence that directs the binding of RNA polymerase and thereby promotes RNA synthesis, and that can comprise one or more enhancers. The promoter can be derived from prokaryotic organisms (such as viruses) and eukaryotic organisms (such as humans). “Enhancers” are cis-acting elements that stimulate or inhibit transcription of adjacent genes. An enhancer that inhibits transcription also is termed a “silencer.” Enhancers differ from DNA-binding sites for sequence-specific DNA binding proteins found only in the promoter (which are also termed “promoter elements”) in that enhancers can function in either orientation, and over distances of up to several kilobase pairs (kb), even from a position downstream of a transcribed region.

The term “constitutive” promoter refers to a promoter which is active under most environmental and developmental conditions.

An “inducible” promoter is a promoter which is under environmental or developmental regulation.

A “tissue specific” promoter is a promoter that is active only in that specific tissue type.

The term “envelope protein” refers to viral envelopes such as VSV-G envelope or other envelope proteins native or modified from GP64, HCV, HBV, LCMV, Rabies virus, Sindbis virus, Sendai virus, Mokola virus, Ebola virus. The envelope protein can be genetically or chemically modified to change its target host range.

The term “expression cassette” refers to a therapeutic gene or reporter gene or any other nucleic acid sequence driven by a promoter and containing a poly-adenylation signal.

The term “poly-adenylation signal” refers to a sequence that is recognized by nucleases that add a polyA tail required for transcript stability and efficient translation into a protein.

The term “fusion protein” refers to a protein containing all or part of the amino acid sequence of two or more proteins in tandem. The fusion of all or part of the coding genetic sequence of the proteins results in the production of the fusion protein.

The term “mutant” refers to a polypeptide that differs from a reference or wild type polypeptide in that it no longer has one or more specific traits or functions present in the reference or wild type polypeptide. A mutant and reference polypeptide may differ in amino acid sequence by one or more substitutions, additions or deletions in any combination. A substituted or inserted amino acid may or may not be one encoded by the genetic code. A mutant of a polypeptide may be naturally occurring or it may not be known to occur naturally.

The term “antisense orientation” or “reverse orientation” refers to the orientation of nucleic acid sequence from a structural gene that is inserted in an expression cassette in an inverted manner with respect to its naturally occurring orientation. When the sequence is double stranded, the strand that is the template strand in the naturally occurring orientation becomes the coding strand, and vice versa.

The term “biologically active nucleic acid sequence” refers to a nucleic acid which has the property of encoding or possessing a property which directs or permits an activity in a host cell. For instance, the nucleic acid optionally encodes proteins or catalytic nucleic acids which act upon the nucleic acid or the host cell upon transcription and/or translation and/or has sequences necessary for chromosomal integration into the host cell's genome, nucleic acid origins of replication, and sequences which permit packaging of the nucleic acid into a viral capsid.

The term “operably linked” refers to functional linkage between a nucleic acid expression control sequence, including, but not limited to, promoter, or array of transcription factor binding sites, or other sequence which functions to control expression, and a second nucleic acid sequence, wherein the expression control sequence directs transcription of the nucleic acid corresponding to the second sequence.

The term “recombinant” when used with reference to a cell indicates that the cell replicates or expresses a nucleic acid, or expresses a peptide or protein encoded by nucleic acid whose origin is exogenous to the cell. Recombinant cells can express genes that are not found within the native (non-recombinant) form of the cell. Recombinant cells can also express genes found in the native form of the cell wherein the genes are re-introduced into the cell by artificial means.

A “nucleic acid” is as previously described. A “nucleic acid sequence” in particular comprises any gene or coding sequence (i.e., DNA or RNA) of potentially any size (i.e., limited, of course, by any packaging constraints imposed by the vector), the possession of which confers a selective advantage, as further defined herein.

A “gene” is any nucleic acid sequence coding for a protein or a nascent mRNA molecule (regardless of whether the sequence is transcribed and/or translated). Whereas a gene comprises coding sequences as well as noncoding sequences (e.g., regulatory sequences), a “coding sequence” does not include any noncoding DNA

The term “identical” in the context of two nucleic acid or polypeptide sequences refers to the residues in the two sequences which are the same when aligned for maximum correspondence. Optimal alignment of sequences for comparison can be conducted, e.g., by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The terms “isolated” or “biologically pure” refer to material which is substantially or essentially free from components which normally accompany it as found in its native state. The isolated nucleic acid vectors of this invention do not contain materials normally associated with their in situ environment, in particular nuclear, cytosolic or membrane associated proteins or nucleic acids other than those nucleic acids intended to comprise the nucleic acid vector itself.

The term “label” refers to a composition detectable by spectroscopic, photochemical, biochemical, immunochemical, or chemical means. For example, useful labels include .sup.32 P, .sup.35 S, fluorescent dyes, electron-dense reagents, enzymes (e.g., as commonly used in an ELISA), biotin, dioxigenin, or haptens and proteins for which antisera or monoclonal antibodies are available.

A “ribozyme sequence” is a catalytic RNA sequence capable of cleaving a target RNA, such as a hairpin or hammerhead ribozyme. The term also encompasses a nucleic acid sequence in an expression cassette from which the RNA is transcribed.

A “miRNA” or microRNA is a single-stranded RNA molecule of about 21- to about 23 nucleotides in length, which regulate gene expression. miRNAs are encoded by genes that are transcribed from DNA but not translated into protein. They are processed from primary transcripts known as pri-miRNA to short stem-loop structures called pre-miRNA and finally to functional miRNA. Their main function is to downregulate gene expression. For that purpose, a miRNA is complementary to a part of one or more messenger RNAs (mRNAs). A miRNA may also target methylation of genomic sites which correspond to targeted mRNAs. Genes encoding miRNAs are much longer than the processed mature miRNA molecule. While miRNA is involved in the normal functioning of eukaryotic cells, dysregulation of miRNA has been associated with disease.

The term “subsequence” in the context of a particular nucleic acid sequence refers to a region of the nucleic acid equal to or smaller than the specified nucleic acid.

According to the invention, a NILV/AAV hybrid vector is introduced into a host cell to achieve a specific biological effect such as to correct a genetic defect, to generate an immune response, to reprogram cell lineages, to knockdown or increase gene expression. The means of introduction comprises contacting a host cell with a vector according to the invention. Preferably, such contacting comprises any means by which the vector is introduced into a host cell; the method is not dependent on any particular means of introduction and is not to be so construed. Means of introduction are well-known to those skilled in the art, and also are exemplified herein.

Accordingly, introduction can be performed, for instance, either in vitro (e.g., in an ex vivo type method) or in vivo, which includes the use of electroporation, transformation, transduction, conjugation or triparental mating, transfection, infection, membrane fusion with cationic lipids, high-velocity bombardment with DNA-coated microprojectiles, incubation with calcium phosphate-DNA precipitate, direct microinjection into single cells, and the like. Other methods also are available and are known to those skilled in the art.

The vectors can be introduced by means of cationic lipids, e.g., liposomes. Such liposomes are commercially available (e.g., Lipofectin®, Lipofectamine™, and the like, supplied by Life Technologies, Gibco BRL, Gaithersburg, Md.). Moreover, liposomes having increased transfer capacity and/or reduced toxicity in vivo (e.g., as reviewed in PCT Patent Application No. WO 95/21259, herein incorporated by reference in its entirety) can be employed in the present invention. In the case of liposome administration, the recommendations identified in PCT Patent Application No. WO 93/23569 can be employed. Generally, with such administration the formulation is taken up by the majority of lymphocytes within 8 hr at 37.degree. C., with more than 50% of the injected dose being detected in the spleen an hour after intravenous administration. Similarly, other delivery vehicles include hydro gels and controlled-release polymers.

Prior to introduction into a host, a vector of the present invention can be formulated into various compositions for use in therapeutic and prophylactic treatment methods. In particular, the vector can be made into a pharmaceutical composition by combination with appropriate pharmaceutically acceptable carriers or diluents, and can be formulated to be appropriate for either human or veterinary applications.

Thus, a composition for use in the method of the present invention can comprise one or more of the aforementioned vectors, preferably in combination with a pharmaceutically acceptable carrier. Pharmaceutically acceptable carriers are well-known to those skilled in the art, as are suitable methods of administration. The choice of carrier will be determined, in part, by the particular vector, as well as by the particular method used to administer the composition. One skilled in the art will also appreciate that various routes (nasal spray, inhalation, intravenous injection, local injection) of administering a composition are available, and, although more than one route can be used for administration, a particular route can provide a more immediate and more effective reaction than another route. Accordingly, there are a wide variety of suitable formulations of the composition of the present invention known to those of skill in the art.

Accordingly, it is an embodiment of the invention to provide a hybrid vector system for targeted integration of a nucleic acid of interest into a host cell comprising of a hybrid transfer vector, an integration defective packaging system, a Vpr-Rep fusion protein and an AAVS1 site.

In another embodiment of the invention, the hybrid transfer vector comprises HIV-1 cis elements, wherein said HIV-1 cis elements required for production and transduction, comprises of a 5′ long terminal repeat (LTR) and a 3′LTR or self-inactivating 3′LTR; a packaging signal (Ψ), central polypurine tract (cPPT) and proximal polypurine tract (PPT).

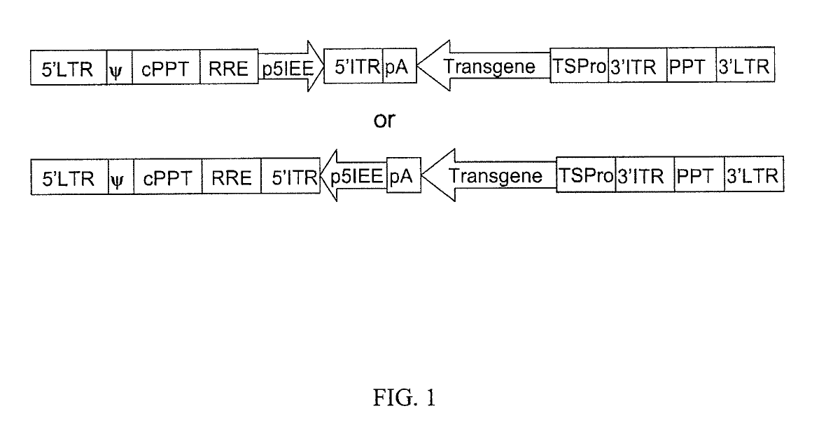

In yet another embodiment of the present invention, the lentiviral vector-derived component of the present invention could be also modified by removing the transcriptional elements of HIV LTR; such as in a so-called self-inactivating (SIN) vector configuration. The modalities of reverse transcription, which generates both U3 regions of an integrated provirus from the 3′ end of the viral genome, facilitate this task by allowing the creation of so-called self-inactivating (SIN) vectors. Self-inactivation relies on the introduction of a disruption (employing for example, deletion, mutation and element insertion) in the U3 region of the 3′ long terminal repeat (LTR) of the DNA used to produce the vector RNA. During reverse transcription, this deletion is transferred to the 5′ LTR of the proviral DNA. If enough sequence is eliminated to abolish the transcriptional activity of the LTR, the production of full-length vector RNA in transduced cells is abolished. This minimizes the risk that RCRs will emerge. Furthermore, it reduces the likelihood that cellular coding sequences located adjacent to the vector integration site will be aberrantly expressed, either due to the promoter activity of the 3′ LTR or through an enhancer effect. Finally, a potential transcriptional interference between the LTR and the internal promoter driving the transgene is prevented by the SIN design. One example of a SIN based lentiviral vector is described in U.S. Pat. No. 6,924,144, the entire contents of which are incorporated herein by reference in its entirety. Non-limiting representative examples of SIN-based lentiviral vectors of the present invention may be generated from one or more of the constructs specifically shown in FIGS. 1, 2, and 9 or any of the other constructs described herein or any combination thereof.

The hybrid transfer vector further comprises AAV-2 cis elements, wherein said AAV-2 cis elements are required for site-specific integration and comprise of 5′ inverted terminal repeats (ITR), and a 3′ITR; p5 integration efficiency element (p5IEE). The p5IEE element is located either 5′ to the 5′LTR in the forward orientation or 3′ to the 5′LTR in the reverse orientation. The hybrid transfer vector further comprises a gene expression cassette, wherein said cassette comprises a nucleic acid sequence of interest operably linked to a functional promoter and a polyA signal. The gene expression cassette is located 3′ to the 5′ITR in the reverse orientation.

In yet another embodiment of the invention, the integration defective packaging system comprises a GagPol containing any or a combination of class I point mutations such as D64V, D116N and E152A in Pol gene to encode an integration defective integrase protein, which converts the hybrid vector virus into non-integrating virus, wherein said GagPol is from HIV-1 virus and is human codon-optimized.

In yet another embodiment of the invention, the Vpr of the Vpr-Rep fusion protein is from HIV-1 and mutated, and the Rep is Rep68/78 and from AAV-2 virus and said directs site-specific integration of transgene flanked by ITRs of AAV-2 at the AAVS1. The AAVS1 site is located on chromosome 19 of human cells. The efficiency of site-specific integration depends on the appropriate ratio of Rep proteins and double-strand DNA templates, as well as the proximity of both in the nucleus. In order to increase the chance of finding the most efficient way for site-specific integration, Rep proteins of AAV-2 can also be alternatively incorporated into LV vector particles through HIV-1 integrase (IN) as IN-Rep fusion protein by fusing Rep protein to the C terminal or N terminal of integrase protein.

In yet another embodiment of the invention, a method for targeted integration of a nucleic acid of interest in a host cell is provided comprising: transducing said host cell with a NILV/AAV hybrid vector comprising of a hybrid transfer vector, an integration defective packaging system, a Vpr-Rep fusion protein and an AAVS1 site. The hybrid transfer vector comprises HIV-1 cis elements, wherein said HIV-1 cis elements required for production and transduction, comprises of a 5′ long terminal repeat (LTR) and a 3′LTR or self-inactivating 3′LTR; a packaging signal (v); central polypurine tract (cPPT) and proximal polypurine tract (PPT). The hybrid transfer vector comprises AAV-2 cis elements, wherein said AAV-2 cis elements are required for site-specific integration and comprise 5′ inverted terminal repeats (ITR), and a 3′ITR; p5 integration efficiency element (p5IEE). The p5IEE element is located either 5′ to the 5′LTR in the forward orientation or 3′ to the 5′LTR in the reverse orientation; and the hybrid transfer vector can further comprise a gene expression cassette, wherein said cassette comprises a nucleic acid sequence of interest operably linked to a functional promoter and a polyA signal. The gene expression cassette is located 3′ to the 5′ITR in the reverse orientation. The integration defective packaging system comprises a GagPol containing any or a combination of class I point mutations such as D64V, D116N and E152A in Pol gene to encode an integration defective integrase protein, which converts the hybrid vector virus into non-integrating virus, wherein said GagPol is from HIV-1 virus and is human codon-optimized. The Vpr of the Vpr-rep fusion protein is from HIV-1 and mutated and said Rep is from AAV-2 virus and directs site-specific integration of transgene flanked by ITR's of AAV-2 at the AAVS1. The AAVS1 site is located on chromosome 19 of human cells.

Referring to the attached drawings, a hybrid vector system based on NILV and AAV vectors is shown in FIG. 1. Such a vector system avoids non-specific integration of LV vector (making LV vectors into nonintegrating LV vectors), and directs site-specific integration of therapeutic gene (flanked by ITRs of AAV) by Rep proteins (Rep68/78) of AAV-2 at AAVS1 site. The AAVS1 site is located on chromosome 19q13.4q. It is about 4 kb long, in open chromatin conformation, and in the vicinity of muscle-specific genes p85, TNNT1, and TNNI3. No known neoplastic malignancies are associated with AAVS1. It may be possible in some circumstances to integrate multiple copies of the ITR-flanked transgene at the AASV1 site.

Moving the Rep gene from the transfer vector into the packaging plasmid and incorporating Rep proteins into HIV particles via Vpr fusion protein provide favorable safety and usability characteristics. Mutations identified in the C-terminal region of Vpr protein and fusing foreign protein to the C-terminal of Vpr protein could potentially eliminate undesirable characteristics of the Vpr protein such as cell cycle arrest and cell killing.

In principle, vector manufacture of NILV/AAV hybrid vector system is based on LV vector production platform, which preserves the advantages of lentiviral vector production, including helper virus-free production, larger packaging capacity (10 kb) and lentiviral vector-mediated gene delivery, including high transduction efficiency in both nondividing and dividing cells.

Still referring to the attached drawings, FIG. 1 illustrates a structure of a LV/AAV hybrid transfer vector in accordance with an example embodiment hereof. In the example, the vector backbone of the transfer vector occupies about 4.5 kb, which leaves about 5.5 kb for the transgene expression cassette. The transgene expression cassette which consists of tissue specific promoter, therapeutic gene or reporter gene and polyA signal, will be located 3′ to the 5′ITR in the reverse orientation. The p5IEE element could be located either 5′to the 5′ITR in the forward orientation or 3′ to the 5′ITR in the reverse orientation. The 5′LTR, Ψ or Psi, cPPT, RRE, PPT and 3′LTR are derived from HIV-1 and are cis elements required for production and transduction. The p5IEE, 5′ITR and 3′ITR are derived from AAV-2 and are cis-elements required for site-specific integration. In a preferred embodiment, no viral protein-coding sequences are present in the transfer vector.

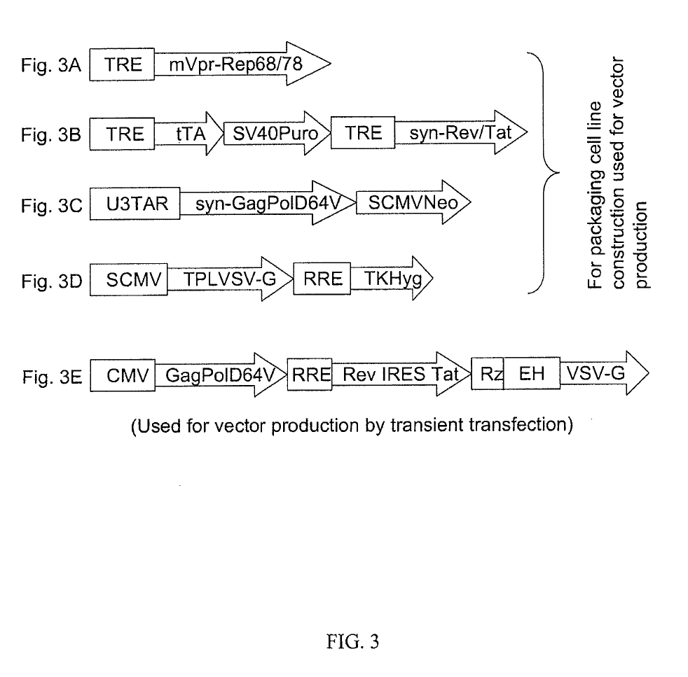

NILV/AAV hybrid vector system comprises four packaging plasmids (FIG. 3A, 3B, 3C and 3D) which are regulated by a tetracycline inducible system and used for packaging cell line construction to produce the hybrid vectors. The two existing packaging plasmids, synRev/Tat plamsid under the control of TRE promoter (FIG. 3B), and VSV-G plasmid driven by a SCMV promoter (FIG. 3D), are used to express Rev/Tat and VSV-G envelop proteins. Rep proteins of AAV-2 are first expressed as a Vpr-Rep fusion protein (FIG. 3A) by TRE promoter, then incorporated into LV vector particles via the Vpr proteins of HIV-1, followed by directing site-specific integration of the transgene at the AAVS1 site within target cells. In an embodiment, introduction of the point mutation D64V in the Pol gene of SynGag-Pol packaging plasmid (FIG. 3C) driven by U3TAR promoter, results in expression and incorporation of a mutant integrase protein into LV vector particles, that does not support integration of LV genomes. Combinations of these functional elements may also be used, such as two or more of these packaging plasmids physically located on one construct.

NILV/AAV hybrid vector system comprises one additional packaging plasmid (FIG. 3E) which contains all viral genes required for production and packaging of hybrid vectors, such as RevTat, GagPolD64V encoding integration defective integrase and VSV-G , is used for producing the hybrid vectors by transient transfection method.

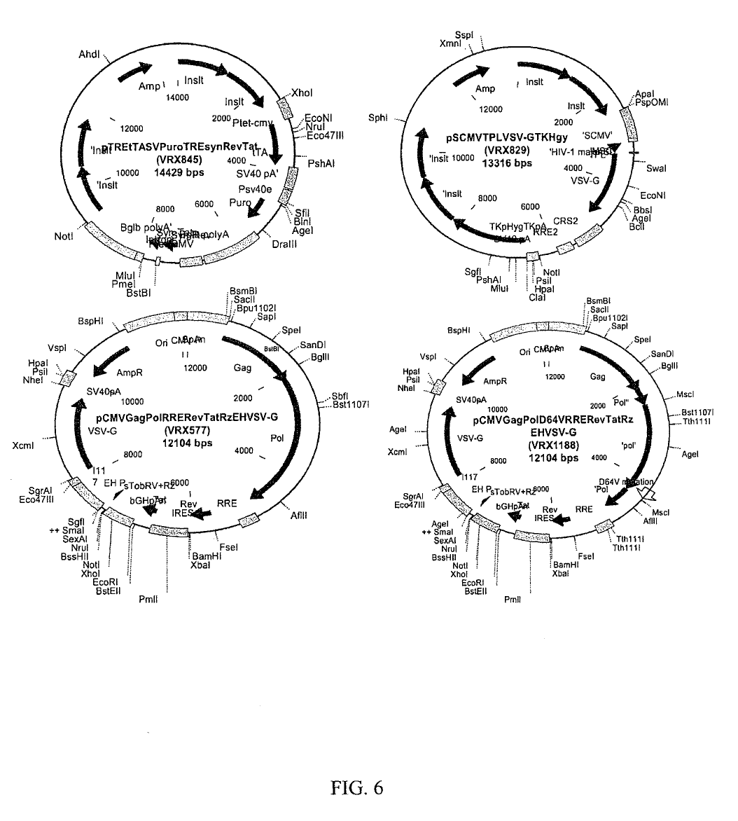

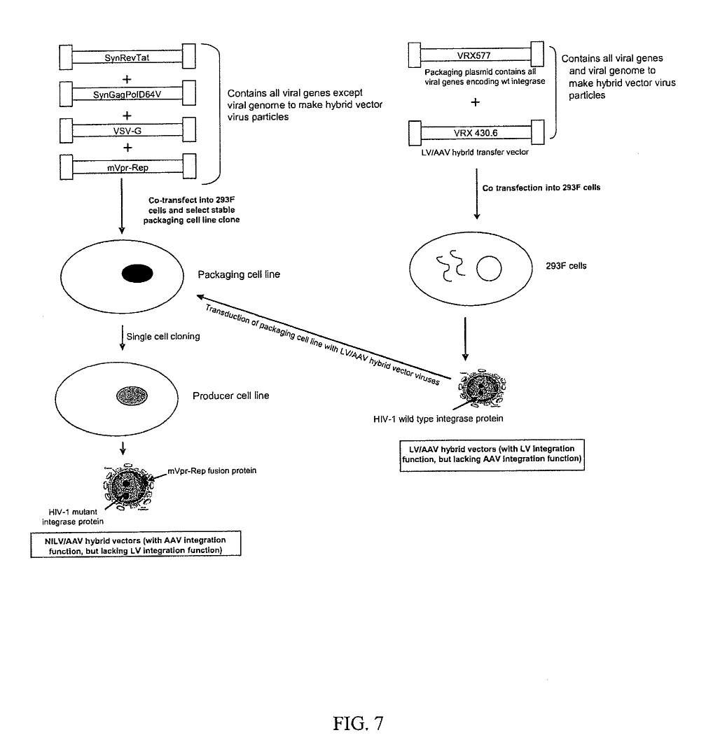

Cell line construction can be carried out using a variety of state of the art-recognized techniques. Example protocols illustrated in FIG. 7 include making an integrating LV/AAV hybrid transfer vector virus using transient cotransfection of the packaging plasmid pCMVGagPolRRERevTatRzEHVSV-G (VRX577, FIG. 6) encoding wild type integrase, together with the hybrid transfer vector plasmid. These integrating LV/AAV vector viruses are used for incorporating the genomes of the hybrid transfer vectors into packaging cell line by transduction.

One skilled in the art will appreciate that numerous equivalents of the foregoing embodiments are readily available and that the following examples may be modified in accordance with the principles hereof using no more than routine experimentation.

### EXAMPLES

### Example 1

### Construction of Hybrid Transfer Vectors

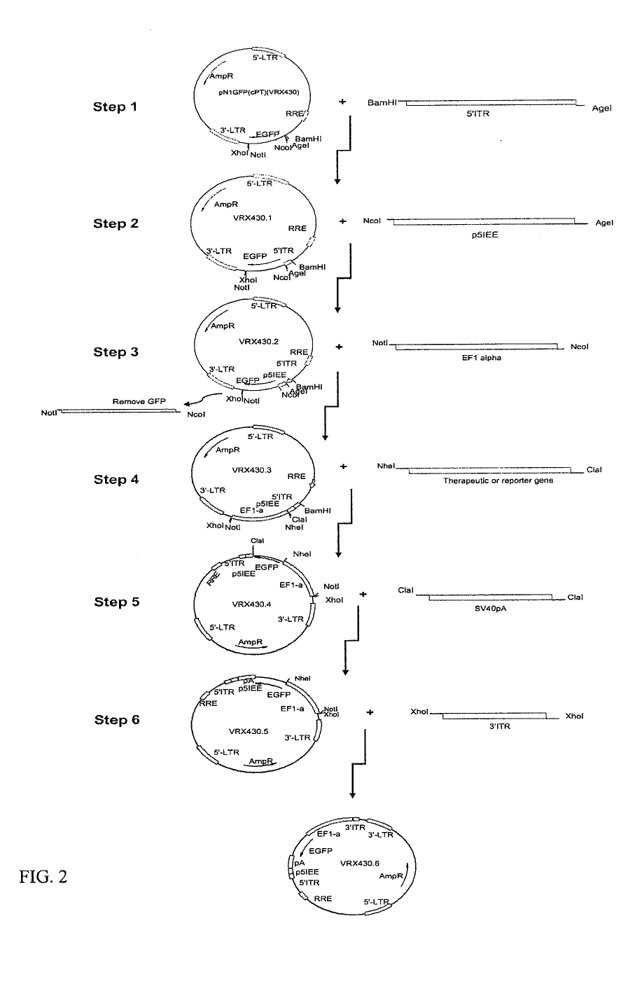

To create a hybrid LV/AAV transfer vector (FIG. 1), cis elements from AAV-2 were incorporated into a LV vector. The HIV-1 cis elements present in the LV vector are the 5′LTR, 3′LTR, packaging signal (Ψ or psi), cPPT, RRE and PPT. The following cis elements derived from AAV-2 were incorporated into LV vector backbone to flank the transgene expression cassette: 5′ITR (145 bp), p5IEE (138 bp), and 3′ITR (145 bp). The transgene expression cassette includes a promoter and a therapeutic or reporter gene and pA signal (FIG. 1). Any of the following LV vectors VRX430, VRX 451 or VRX448, which contain the necessary aforementioned HIV-1 cis elements were used as to create the hybrid LV/AAV transfer vector, depending on cloning convenience. An example of one of several cloning strategies that can be used to create the hybrid LV/AAV transfer vector is provided (FIG. 2). In the first step, chemically synthesized 5′ITR of AAV2 was ligated into LV vector VRX430 at the BamHI and AgeI sites to create intermediate construct VRX430.1. The chemically synthesized p5IEE element of AAV2 was ligated into the NcoI and AgeI site of VRX430.1 to create intermediate construct VRX430.2. EF1 alpha or a tissue specific promoter was ligated into the NotI and NcoI site of VRX430.2 in the reverse orientation, while simultaneously removing GFP reporter gene, to create intermediate construct VRX430.3. The therapeutic gene or a reporter gene was ligated into the NheI and ClaI site upstream of the promoter in the reverse orientation, to create intermediate construct VRX430.4. The SV40 polyA signal was ligated upstream of the therapeutic or reporter gene in the ClaI site, to create intermediate construct VRX430.5. The synthesized 3′ITR of AAV2 was ligated in the XhoI site, to create the final hybrid LV/AAV transfer vector construct (FIG. 2).

### Example 2

### Construction of Packaging Constructs

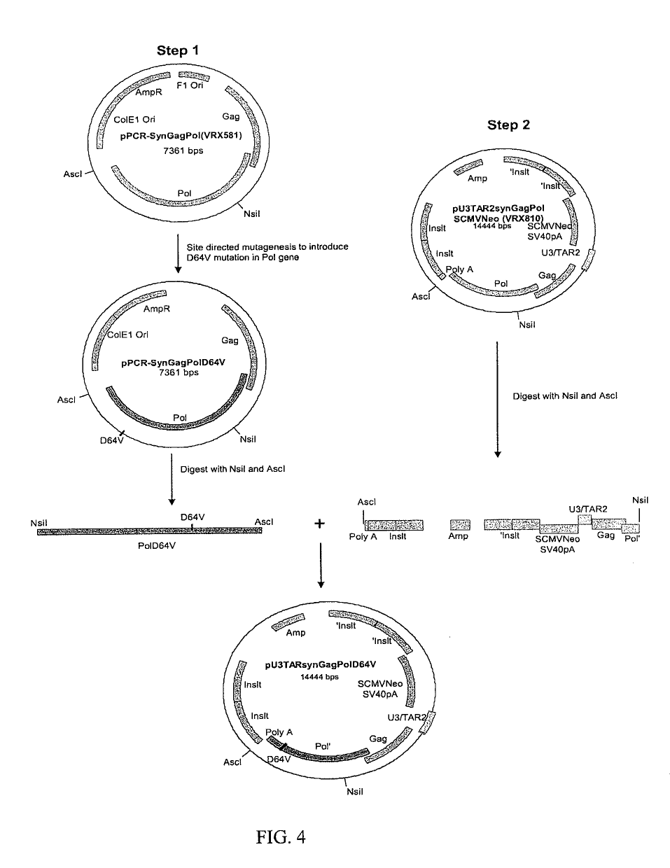

The packaging constructs necessary for the packaging and production of the NILV/AAV hybrid vector virus contains HIV-1 elements and AAV-2 Rep68/78 protein provided in trans (FIG. 3). The packaging construct pTREtTASVpuroTREsynRevTat (VRX845) provides Rev and Tat proteins from a tetracycline inducible system. The pSCMVTPLVSVGTKHyg (VRX829) construct provides the envelope protein VSVG. The two constructs VRX845 and VRX829 are pre-existing (FIG. 6). The packaging construct pU3TARsynGagPolD64V (FIG. 4) were constructed from the pre-existing vector pU3TARsynGagPolSCMVNeo (VRX810), which provides Gag and Pol proteins. The integrase protein encoded by Pol gene in VRX810 was a wild type protein. In order to make the integration-defective integrase, a point mutation D64V has to be introduced in the Pol gene of VRX810 to create pU3TARsynGagPolD64V. The D64V mutation is introduced into the Pol gene of construct pPCR-synGagPol (VRX581) by site directed mutagenesis. The region of the Pol gene containing the D64V mutation is cut out using AscI-NsiI and ligated in the AscI-NsiI site of VRX810 to replace the wt region of Pol to create integration defective packaging construct pU3TARsynGagPolD64V (FIG. 4).

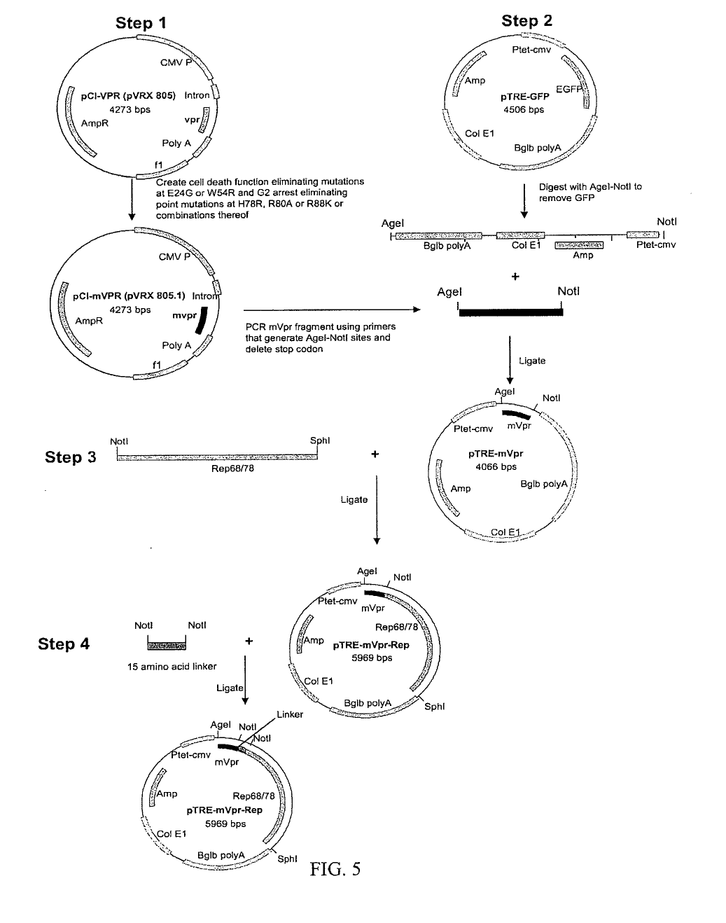

For the pTRE-mVpr-Rep plasmid (FIG. 5), the following point mutations can be introduced into wild type Vpr protein singly or in combination to abort G2 arrest and cell killing effects while maintaining nuclear localization and virion packaging capacities of Vpr protein. G2 arrest point mutations: H78R, R80A and R88K; cell killing point mutations: E24G and W54R. The full Rep gene from AAV-2 (only allowing expression of Rep68/78 but not Rep 40/52) was fused to the C-terminus of wild type or mutant Vpr protein. The Vpr-Rep fusion protein was cloned downstream of the TRE promoter in the pTRE2 (Clontech) plasmid. A selection marker gene such as zeocin can be added into the pTRE-mVpr-Rep plasmid (FIG. 5).

### Example 3

### Construction of Packaging Cell Line and Hybrid Vector Producer Cell Line

The packaging plasmids, for example, pTREtTAsynRevTatSV40Puro (VRX845), pSCMVTPLVSV-GTKHyg (VRX829), pTRE-mVpr-Rep, and pU3TARsynGagPoID64V, were simultaneously incorporated into either 293F cells, 293 cells, 293T cells, PerC6 cells, or other human cells, by cotransfection followed by single cell cloning to establish a packaging cell line (FIG. 7). LV/AAV hybrid vector viruses made by transient cotransfection of the packaging plasmid pCMVGagPolRRERevTatRzEHVSV-G (VRX577, FIG. 6) encoding a wild type integrase and the hybrid transfer vector can be used to transduce packaging cells at appropriate moi to incorporate the genome of hybrid transfer vector into cell chromosomes. Producer cell lines containing LV/AAV transfer vector were then established by single cell cloning. (FIG. 7).

### Example 4

### Hybrid NILV/AAV Vector Production by Two Different Methods

### Transient Transfection and Producer Cell Line

NILV/AAV hybrid vectors can be produced by transient cotransfection of the packaging plasmids pCMVGagPolD64VRRERevTatRzEHVSV-G (VRX1188, FIG. 6) encoding an integration defective integrase and pTRE-mVpr-Rep (FIG. 5), together with the hybrid transfer vector (FIG. 2), using 293F cells. Alternatively, NILV/AAV hybrid vectors can also be produced by the established producer cell line described above in FIG. 7.

### Example 5

### Determining Efficiency of Transduction and Site-Specific Integration of Hybrid Vectors

293T, Hela-tat or related tissue-specific cell lines can be used for monitoring transduction efficiency. The target cells were seeded at an appropriate cell number to obtain ˜50% confluency and transduced with the NILV/AAV hybrid vector virus at different moi. The transduction efficiency was determined using vector copy number assessment by Quantitative PCR, transgene expression levels and site-specific integration using LM-PCR or Southern Blot.

### Example 6

### Examination of G2 Arrest and Toxicity of VprRep Fusion Protein in Target Cells after Hybrid Vector Transduction

Cells transduced with hybrid vectors were subjected to cell cycle analysis by flow cytometry and cell survival measurement to assess the level of G2 arrest and cell killing of Vpr fusion protein.

### Example 7

### Gene Delivery by Hybrid Vector into Dividing Cells

Therapeutic genes carried by the hybrid vectors were delivered into hematopoietic stem cells ex vivo to treat genetic diseases such as hemophilia A, anemia, infectious diseases such as HIV, cancers such as leukemia or lymphoma, Alzheimer's disease among others. The therapeutic gene or transgene can include cDNA molecules, antisense RNA molecules, pre-RNA molecules, miRNA molecules, siRNA molecules, ribozyme molecules, suicide molecules, genes coding for therapeutic proteins, enzymes, and antibodies, among others.

### Example 8

### Local or Systematic Gene Delivery by Hybrid Vectors into Various Organs and Tissues

Hybrid vectors are directly delivered into brain, liver, inner ear, retina, bladder, prostate, and breast to treat Parkinson's disease, Alzheimer's disease, dislipidemia, hemophilia A, HCV infection, inner ear disorders, ocular diseases, bladder cancer, breast cancer, and prostate cancer, among others. The gene or trasngene may include cDNA molecules, antisense RNA molecules, pre-RNA molecules, mRNA molecules, siRNA molecules, ribozyme molecules, suicide molecules, genes coding for therapeutic proteins, enzymes, and antibodies, among others.

### Example 9

### Induction of Pluripotent Stem Cells from Normal or Diseased Fibroblasts

Hybrid vectors are used to make induced pluripotent stem cells (iPS) from normal or diseased adult somatic cells, and correct the gene defect in diseased iPS. These iPS can then be used to regenerate normal tissue or the patient or disease-specific tissue or organ to treat various diseases.

### Example 10

### NILV/AAV Hybrid Vectors for Sickle Cell Anemia

NILV/AAV vectors expressing the wild type β-globin gene by the EF-1α, PGK or Ubiquintin or other constitutive promoter, are produced and concentrated. Mobilized peripheral blood from sickle cell anemia patient is collected till CD34+ counts reach 2−10×10⁶/kg body weight. CD34+ hematopoietic stem cells are isolated using CliniMACS CD34 magnetic bead separation. CD34+ cells are transduced with NILV/AAV 13-globin gene at moi of 1-5 for 12-24 hr in serum free XVivo-10 media containing cytokines, human stem cell factor (hSCF), human thrombopietin (hTPO), human Flt3 and human II-3. The transduced cells are washed and resuspended in sterile saline and reinfused into the patients at 2−10×10⁶ CD34+ cells/kg body weight.

In Examples 11, 12, and 13 infra, a specific reference is made to pre-transplicing molecules (PTMs). PTMs comprise a target binding domain that is designed to specifically bind to an endogenous pre-mRNA, a 3′ splice region that includes a branch point, pyrimidine tract and a 3′ splice acceptor site and/or a 5′ splice donor site; and a spacer region that separates the RNA splice site from the target binding domain. In addition, PTMs can be engineered to contain nucleotide sequences encoding any protein of interest or a fragment thereof, which upon trans-splicing, produces a functional protein. A full description of the generation and use of such PTMs for correction of endogenous mRNAs to yield expression of corrected proteins and/or heterologous proteins is described in more detail in Applicants co-pending U.S. Patent Publication No. US 2006-0194317 A1 (the contents of which are incorporated herein by reference in their entirety).

### Example 11

### NILV/AAV Hybrid Vectors for Treatment of Type 1 and Certain Type 2 Diabetes Patients

Primary skin fibroblasts cultures are established from the patient's skin biopsy sample and cultured for 7-14 days. 5×10⁴ primary fibroblast are transduced overnight with a combination of four integrating or non-integrating lentiviral vectors containing the pre-transplicing molecule (PTM) or cDNAs for Oct4, Sox2, NANOG, LIN28 or Oct3/4, Sox2, klf4, c-myc or any combination of the 6 genes at 2.5×10⁵ to 2.5×10⁶ transducing units of each vector, for 12-16 hr. Alternatively, by using bidirectional constitutive or tissue specific promoters, each vector will express two of the PTM's or cDNA's. The lentiviral vectors also have LoxP sites flanking the PTM's or cDNAs which can later be excised with a Cre recombinase expressing vector. This adds an additional safety feature that ensures removal of the vector after reprogramming to the embryonic stage is achieved. The transduced fibroblasts are cultured for 6 days in DMEM with 10% serum followed by plating 5×10⁴ cells per 100 mm dish on feeder cell layer. The cells are cultured for an additional 30 days in human ES media containing 4 ng/ml basic fibroblast growth factor (bFGF). iPS colonies are isolated and expanded on feeder cells or in feeder free conditions on Matrigel coated plates. The colonies are transduced with a Cre expressing vector to excise the PTM's, and then expanded for 5-10 passages. ˜10-20 million iPS cells are frozen down and banked. ˜1−10×10⁸ iPS are transduced with a NILV/AAV hybrid lentiviral vector expressing human insulin gene and/or GLP-1 at moi of 5-10, for 12-16 hrs. The vector will then be washed off and the cells are allowed to grow for a further 48-72 hrs. in order to start expressing the gene. iPS are directed to differentiate along the endoderm lineage into pancreatic endodermal cells using growth factors activin, Wnt, KAAD-cyclopamine, human fibroblast growth factor 10 (FGF-10), retinoic acid, γ secretase inhibitor, extendin, insulin growth factor 1 (IGF1), hepatocyte growth factor 1 (HFG1), FBS, and growth supplements. 1×10⁸-1×10⁹ insulin secreting cells/kg body weight or 10,000 islet equivalents/kg body weight are injected via a catheter inserted through the upper abdomen and guided to the liver via the hepatic portal vein. An alternate protocol involves growth and encapsulation of insulin secreting cells in Gelfoam and Matrigel or other suitable artificial biomembrane material, and implantation into the recipient. Implants are done either in subcutaneous adipose tissue or under the kidney capsule.

### Example 12

### NILV/AAV Hybrid Vectors for Cystic Fibrosis

NILV/AAV hybrid vectors expressing the PTM or cDNA for the wt CFTR protein from a constitutive (EF-1α, PGK or Ubiquitin) or a tissue specific promoter are produced from the packaging cell line and concentrated to 5×10⁸-1×10⁹ TU/ml. Delivery and persistence of the vector particles are enhanced by a viscoelastic gel system such as methylcellulose in which the vector particles are enmeshed. Vector particles are prepared in 1% methylcellulose or other FDA approved gel system, at concentration of 1×10⁵-1×10⁶ TU/μl. Vector is administered intra-nasally at 1×10⁸-1×10⁹ TU in either one or in both nostrils. An alternate delivery route could be intra-esophageal and would be required to be performed under local anesthesia.

### Example 13

### NILV/AAV Hybrid Vectors for Parkinson's Disease

NILV/AAV2 hybrid vectors expressing the PTM or cDNA for Glial cell-derived neurotrophic factor (GDNF) or Aromatic L-amino acid decarboxylase (AADC) are produced from the packaging cell line and concentrated to 5×10⁸-1×10¹⁰ TU/ml. Two days before surgery, the patient is given an MRI scan while wearing an MRI-compatible stereotactic frame. This is done to obtain images for precise stereotactic cannula insertion. Before surgery, the patient is sedated and placed in a stereotactic frame. A bone flap is made in the skull to expose the dura at the exact target site. Cannulae are guided into the brain with coordinates generated by the MRI. Two to six sites are chosen for vector injection. Vector is administered via the cannulae at rates ranging from 0.1/min to 1 μA/min at volumes of 50-500 μl per injection site. After injection, the cannulae are raised until clear of the brain. Patients are monitored for recovery.

### Example 14

### NILV/AAV Hybrid Vectors for Retinitis Pigmentosa

Retinitis pigmentosa (RP) is a heterogeneous group of inherited eye disorders that are characterized by progressive degeneration of the rod and cone photoreceptor cells. Several genes have been found to be associated with retinal degeneration, including mutations in the rod photoreceptor cGMP phosphodiesterase β subunit (PDEβ) gene, in autosomal recessive RP. Leber's congenital amaurosis (LCA) is a group of autosomal recessively inherited dystrophies that are severe infantile onset degeneration of the rod and cone receptors. LCA2 is caused by mutations in the retinal pigment epithelial specific 65 kD protein gene (RP65). This protein is required for producing 11-cis-retinal that is required for light capture by the rod and cone photoreceptors, and the absence of the functional protein results in loss of vision and eventual degeneration of retinal cells.

No cure exists for these groups of disorders. NILV/AAV2 hybrid vectors are used to locally deliver the therapeutic gene. Vectors expressing either PDEβ gene or the RP65 gene are produced and concentrated to 1×10⁹-1×10¹⁰ TU/ml. The genes are expressed either from a constitutive promoter or a tissue specific promoter such as rhodopsin that will allow expression only in the photoreceptor cells. The vectors are injected into the sub-retinal space using standard sub-retinal surgery techniques, while patients are under general anesthesia. The vector to be injected is adjusted to 1×10⁸ to 1×10⁹TU/100 μl. 150 μl of the vector is injected into the sub-retinal space. Patients are given prednisone to control inflammation.

### Example 15

### NILV/AAV Hybrid Vectors for Drug Screening and Discovery

Target-based drug screening and development uses cells that have been engineered to have dysregulated expression of genes associated with particular diseases. Traditional methods to overexpress genes use a strong promoter to express the cDNA of the gene which is then transfected into a cell line and stable clones selected for high expression. Gene dysfunction is also manifested as under-expression or no expression of the particular gene. This can be achieved in vitro by stably transfecting ribozymes, antisense molecules or microRNA's into a model cell line such as CHO cells, HepG2 cells, Hela cells or other mammalian cells (preferably human or mouse). Stable expression can also be achieved by using LV vectors or retroviral vectors (RV) to stable integrate the gene or molecule into cells. Such engineered cells can be used to screen potential therapeutic compounds. The disadvantage of using DNA transfection methods or LV or RV vectors as gene delivery vehicles is random insertion of the expression cassette into the cellular genome, contributing to potential insertional mutagenesis events, transgene silencing and unpredictable gene expression levels, all of which could influence the validity of the screen.

Use of NILV/AAV hybrid vectors to deliver the cDNA or siRNA or microRNA or ribozyme of interest could avoid potential problems associated with random integration events, and achieve site specific integration into the AAVS1 site on chromosome 19. This site is not known to be associated with any tumorigenic or mutagenic event. Furthermore single integration events can be achieved thus ensuring uniform and controlled expression levels. The expression of the transgenes can also be made regulatable by using inducible promoters.

The cDNA, microRNA, siRNA or ribozyme of interest expressed in the NILV/AAV hybrid vector can be produced and used to transduce a model cell type. Cell clones stably expressing the gene can be selected and tested for transgene expression levels, site-specific integration and integrated vector copy number. Single cells clones can be expanded and used for screening of candidate drug molecules.

### Example 16

### NILV/AAV Hybrid Vectors for Bioengineering

NILV/AAV vectors could be used as a gene delivery vehicle for generating cell banks for manufacture of therapeutic proteins. NILV/AAV hybrid vectors expressing biologics such as gene therapy vectors, vaccines, blood components or therapeutic proteins and a drug resistance gene, could be used to transduce cells. Stably transduced clones can be selected using drug selection and single cell cloning. Producer cell clones can be screened for therapeutic protein expression levels, site specific integration and vector copy number. Site specific integration of the NILV/AAV hybrid vector occurs into the AAVS1 site on chromosome 19 that is not known to be associated with malignant transformation, ensuring a producer cell line that will not be tumorigenic or produce oncogenic by-products. The therapeutic proteins if secreted can be purified from the media by affinity chromatography, ion-exchange chromatography, HPLC or other such procedures known to those skilled in the art. If the therapeutic protein is intracellular, it can be released by standard procedures known to those skilled in the art, such as homogenization and/or sonication followed by centrifugation and immunoprecipitation.

### Example 17

### Stable Gene Expression from LV-AAV Hybrid Vector Plasmid in Hela-Tat Cells Resulting from Rep-Mediated Integration

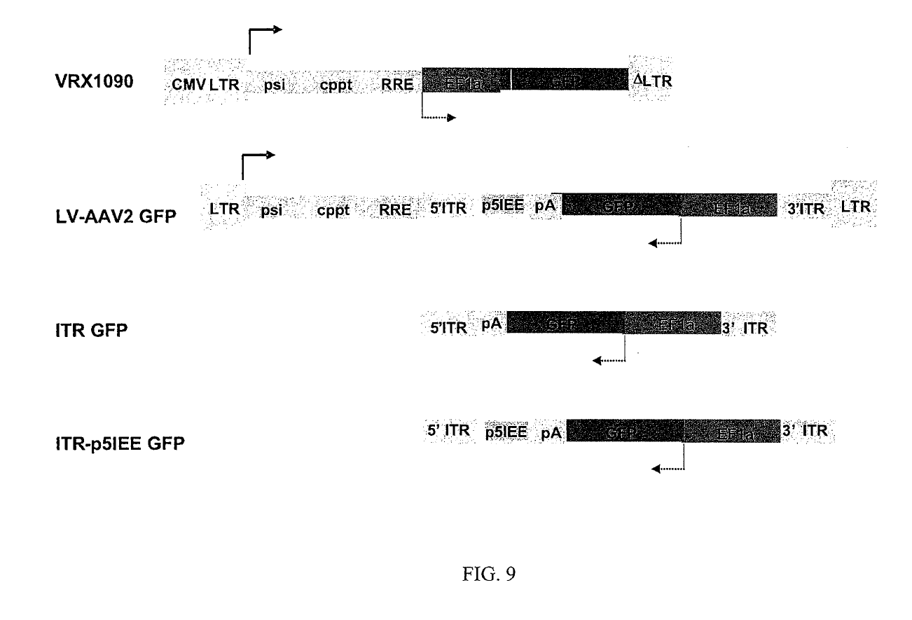

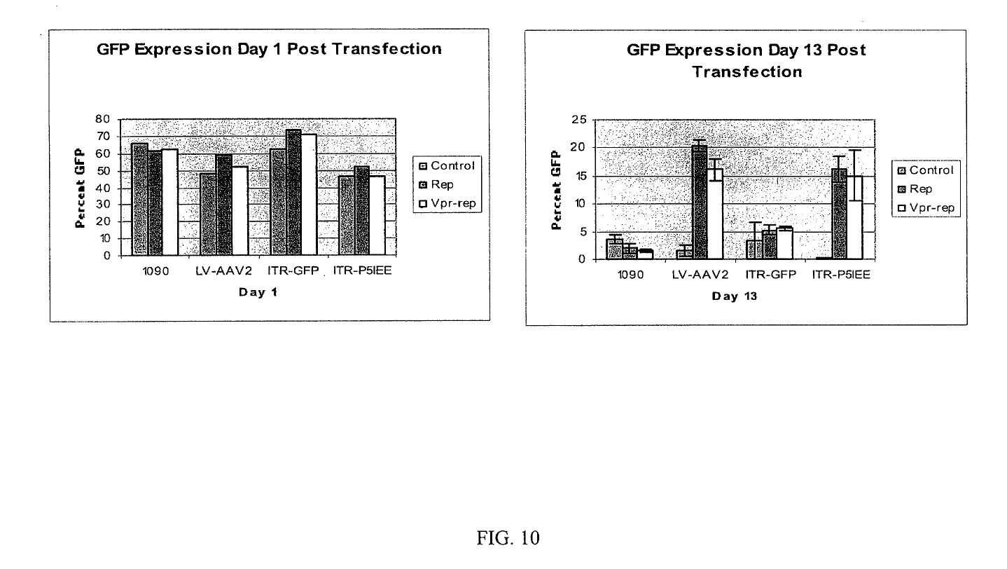

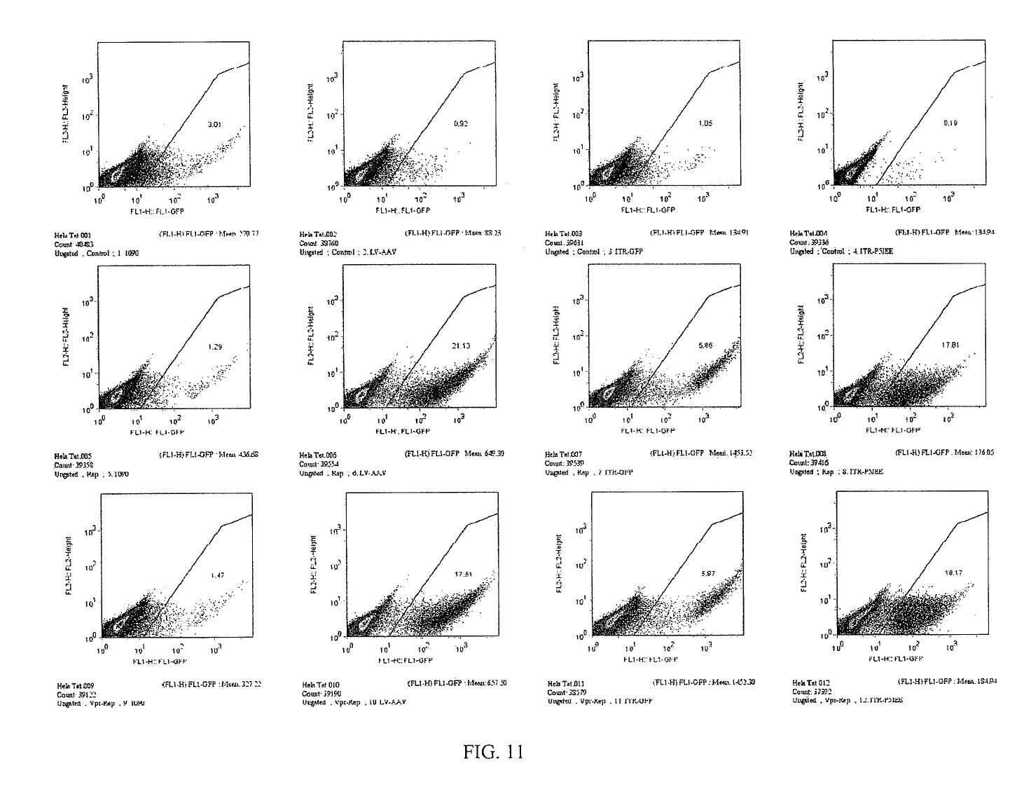

An experiment was conducted to examine whether Rep and Vpr-Rep proteins can recognize AAV-2 cis elements (ITRs and p5IEE) present in LV vector backbone and mediate site-specific integration. The related plasmids used for the experiment are shown in FIG. 9. Hela-tat cells were cotransfected with VRX1090 control, LV-AAV2 GFP, ITR-GFP, or ITR-p5IEE-GFP plasmid along with control mock, Rep, or Vpr-Rep plasmid. GFP expression was determined on day 1, day 6, day 13 and day 16 after transfection by flow cytometry analysis. As shown in FIG. 10 and FIG. 11, less than 2% stable gene expression resulted from random integration occurred in VRX1090 (which is a control GFP expressing SINLV construct) plasmid on day 13, but in the plasmids containing ITR and/or p5IEE stable gene expression resulted from Rep-mediated integration was increased nearly 10-fold to about 15%-20%. When either p5IEE or Rep was removed from the cotransfection there was a dramatic loss in stable gene expression resulted from integration. These data indicate Vpr-Rep fusion protein functions as well as Rep protein, and efficiently mediates integration of ITR-flanked GPF expression cassette from LV vector backbone and results a functional yet stable gene expression. However, the site-specific integration at AAVS1 site of chromosome 19 and its frequency need to be examined and confirmed by quantitative PCR and southern blot analysis in the stably transfected hela-tat cells in the future.

The foregoing description of some specific embodiments provides sufficient information that others can, by applying current knowledge, readily modify or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. In the drawings and the description, there have been disclosed exemplary embodiments and, although specific terms may have been employed, they are unless otherwise stated used in a generic and descriptive sense only and not for purposes of limitation, the scope of the claims therefore not being so limited. Moreover, one skilled in the art will appreciate that certain steps of the methods discussed herein may be sequenced in alternative order or steps may be combined. Therefore, it is intended that the appended claims not be limited to the particular embodiment disclosed herein.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims; or in the text itself; and, each of these documents or references (“herein-cited references”), as well as each document or reference cited in each of the herein-cited references (including any manufacturer's specifications, instructions, etc.), is hereby expressly incorporated herein by reference.

What is claimed is:

1. A hybrid vector system for targeted integration of a nucleic acid of interest into a host cell comprising of a hybrid transfer vector, an integration defective packaging system, a Vpr-Rep fusion protein and an AAVS1 site.

2. The hybrid vector system of claim 1, wherein said hybrid transfer vector comprises HIV-1 cis elements, wherein said HIV-1 cis elements required for production and transduction, comprises of a 5′ long terminal repeat (LTR) and a 3′LTR or self-inactivating 3′LTR; a packaging signal (Ψ), central polypurine tract (cPPT) and proximal polypurine tract (PPT).

3. The hybrid vector system of claim 1, wherein said hybrid transfer vector comprises AAV-2 cis elements, wherein said AAV-2 cis elements are required for site-specific integration and comprise of 5′ inverted terminal repeats (ITR), and a 3′ITR; p5 integration efficiency element (p5IEE).

4. The hybrid vector system of claim 3, wherein the said p5IEE is located either 5′ to the 5′LTR in the forward orientation or 3′ to the 5′LTR in the reverse orientation.

5. The hybrid vector system of claim 1, wherein said hybrid transfer vector comprises a gene expression cassette, wherein said cassette comprises a nucleic acid sequence of interest operably linked to a functional promoter and a polyA signal.

6. The hybrid vector system of claim 5, wherein said gene expression cassette is located 3′ to the 5′ITR in the reverse orientation.

7. The hybrid vector system of claim 1, wherein said integration defective packaging system comprises GagPol, wherein said GagPol contains any or a combination of class I point mutations such as D64V, D116N and E152A in Pol gene to encode an integration defective integrase, which converts the hybrid vector virus into non-integrating virus, wherein said GagPol is from HIV-1 virus and is human codon-optimized.

8. The hybrid vector system of claim 1, wherein said Vpr-Rep fusion protein comprises Vpr and Rep proteins, wherein said Vpr is from HIV-1 and mutated, wherein said Rep is Rep68/78 and from AAV-2, and said directs site-specific integration of transgene flanked by ITRs of AAV-2.

9. The hybrid vector system of claim 1, wherein said AAVS1 site is present on chromosome 19 of human cells.

10. A method for targeted integration of a nucleic acid of interest in a host cell comprising:
transducing said host cell with a NILV/AAV hybrid vector comprising of a hybrid transfer vector, an integration defective packaging system, a Vpr-Rep fusion protein and an AAVS1 site.

11. The method of claim 10, wherein said hybrid transfer vector comprises HIV-1 cis elements, wherein said HIV-1 cis elements required for production and transduction, comprises a 5′ long terminal repeat (LTR) and a 3′LTR or self-inactivating 3′LTR; a packaging signal (v); central polypurine tract (cPPT) and proximal polypurine tract (PPT), or any combination thereof.

12. The method of claim 10, wherein said hybrid transfer vector comprises AAV-2 cis elements, wherein said AAV-2 cis elements are required for site-specific integration and comprise 5′ inverted terminal repeats (ITR), and a 3′ITR; p5 integration efficiency element (p5IEE), or any combination thereof.

13. The method of claim 12, wherein the said p5IEE is located either 5′ to the 5′LTR in the forward orientation or 3′ to the 5′LTR in the reverse orientation.

14. The method of claim 10, wherein said hybrid transfer vector comprises a gene expression cassette, wherein said cassette comprises a nucleic acid sequence of interest operably linked to a functional promoter and a polyA signal.

15. The method of claim 14, wherein said gene expression cassette is located 3′ to the 5′ITR in the reverse orientation.

16. The method of claim 10, wherein said integration defective packaging system comprises GagPol, wherein said GagPol contains any or a combination of class I point mutations such as D64V, D116N and E152A in Pol gene to encode an integration defective integrase, which converts the hybrid vector virus into non-integrating virus, wherein said GagPol is from HIV-1 virus and is human codon-optimized.

17. The method of claim 10, wherein said Vpr-Rep fusion protein comprises Vpr and Rep proteins, wherein said Vpr is from HIV-1 and mutated, wherein said Rep is Rep68/78 and from AAV-2 virus and said directs site-specific integration of transgene flanked by ITR of AAV-2.

18. The method of claim 10, wherein said AAVS1 site is present on chromosome 19 of human cells.
